(19) United States
(12) Patent Application Publication
(10) Pub. No.: US 2022/0095927 A1
(43) Pub. Date: Mar. 31, 2022

(54) PHOTOACOUSTIC DEVICE

(71) Applicant: UCL Business Ltd, London (GB)

(72) Inventors: Edward Zhang, London (GB); Paul Beard, London (GB); Rehman Ansari, London (GB); Nam Trung Hunynh, London (GB)

(21) Appl. No.: 17/426,469

(22) PCT Filed: Jan. 30, 2020
(86) PCT No.: PCT/GB2020/050216

§ 371 Date: Jul. 28, 2021

(30) Foreign Application Priority Data
Feb. 1, 2019 (GB) 1901453.9

(51) Int. Cl. A61B 5/00; A61B 1/24; A61B 1/00; A61B 1/04

(52) U.S. Cl. CPC A61B 5/0095; A61B 1/24; A61B 1/00096; A61B 1/04; A61B 1/00172; A61B 1/00165

(57) ABSTRACT

A photoacoustic probe head (200a, 200b) is disclosed. The probe head (200a, 200b) comprises: a Fabry Perot acoustic sensor (202), an interrogation interface (A), an excitation input (B) and a two-axis mirror (204). The Fabry Perot acoustic sensor (200a, 200b) is operable to reflect an optical interrogation beam to create a reflected interrogation beam and to modulate the reflected interrogation beam in response to an acoustic signal at the acoustic sensor (202). The interrogation interface (A) is configured to receive an interrogation beam, and to receive the reflected interrogation beam from the acoustic sensor (202). The excitation input (B) is configured to receive an excitation beam for generating an acoustic field in a sample adjacent to the acoustic sensor (202). The two axis mirror (240) is configured to scan the interrogation beam between different locations of the acoustic sensor (202).

### TECHNICAL FIELD

The present invention relates to a method and apparatus for performing photoacoustic imaging.

### BACKGROUND

In photoacoustic (PA) imaging, acoustic waves are excited by irradiating a sample with modulated electromagnetic radiation. The wavelength of the electromagnetic radiation is typically in the optical or near infrared band. In general, the longer wavelengths allow greater penetration of the radiation into the tissue, of the order of several centimetres. The radiation is then absorbed within the tissue which leads to a slight rise in temperature, typically about 0.1K, which is sufficiently small to avoid any physical damage or physiological change to the tissue. The rise in temperature causes the emission of ultrasound (acoustic) waves, which are broadband (approximately tens of MHz) and relatively low amplitude (less than 10 kPa). These ultrasound waves propagate to the surface of the tissue, where they can be detected by a suitable mechanism, for example, a single mechanically scanned ultrasound receiver or an array of receivers.

A Fabry Perot acoustic sensor may be used to detect the acoustic waves. Such devices typically employ a polymer film etalon that comprises a polymer film spacer sandwiched between a pair of mirrors. Acoustically induced changes in the optical thickness of the spacer modulate the reflectivity of the etalon, which can be detected by measuring the changes in reflected power of an incident interrogation beam. By scanning a focussed interrogation beam across the surface of the sensor, an incident PA wavefront can be spatially mapped in two dimensions.

There are several advantages of this concept. First, the mirrors of the etalon can be designed to be transparent to the excitation laser wavelength. The sensor head can therefore be placed directly on the surface of the skin, and the laser pulses transmitted through it into the underlying tissue. It thus avoids the detection aperture limitations imposed by an acoustic spacer required for piezoelectric detection methods. It also provides an inherently broadband response from DC to several tens of megahertz and very fine spatial sampling of the incident acoustic field. The effective acoustic element size is defined, to a first approximation, by the diffraction limited dimensions of the focused interrogation laser beam. The notional element size and inter-element spacing can therefore be on a scale of tens of micrometres. Perhaps, most importantly, the small element size is achieved with significantly higher detection sensitivity that can be provided by similarly broadband piezoelectric receivers of the same element dimensions.

Although considerable progress has been made in the development of PA imaging systems, challenges still remain. For example, a more compact and flexible PA imaging system would be advantageous.

### SUMMARY

According to a first aspect of the invention, there is provided a photoacoustic probe head, comprising:
a Fabry Perot acoustic sensor, operable to reflect an optical interrogation beam to create a reflected interrogation beam and to modulate the reflected interrogation beam in response to an acoustic signal at the acoustic sensor;
an interrogation interface configured to receive an interrogation beam, and to receive the reflected interrogation beam from the acoustic sensor;
an excitation input configured to receive an excitation beam for generating an acoustic field in a sample adjacent to the acoustic sensor;
a two-axis mirror configured to scan the interrogation beam between different locations of the acoustic sensor.

The two-axis mirror may be a MEMS mirror (in any aspect of the invention).

The two-axis mirror may be configured to direct the reflected interrogation beam to the interrogation interface.

The photoacoustic probe head may further comprise:
a mirror housing within which the mirror is disposed, and
a sensor housing with the acoustic sensor at a distal tip thereof,
wherein the mirror housing and sensor housing together comprise a mechanical coupling for removably attaching the sensor housing to the mirror housing.

The acoustic sensor may be coupled to a refracting prism, the refracting prism configured to deflect the interrogation beam to provide an offset viewing direction of the acoustic sensor.

According to a second aspect, there is provided a photoacoustic imaging apparatus, comprising:
the probe head according to the first aspect,
an interrogation light source configured to generate the interrogation beam and coupled to the interrogation input;
an excitation light source configured to generate an excitation beam for generating an acoustic field in a sample adjacent to the acoustic sensor, the excitation light source coupled to the excitation input;
a light detector coupled to the interrogation port and configured to detect the reflected interrogation beam from the acoustic sensor;
a controller configured to:
   use the excitation light source to generate an acoustic field in the sample, and
   detect the acoustic field at a plurality of locations of the acoustic sensor by scanning the mirror and using the light detector.

The probe head may be connected to the interrogation light source and the excitation light source by a flexible umbilical.

According to a third aspect, there is provided a photoacoustic imaging apparatus, comprising:
a Fabry Perot acoustic sensor, operable to reflect an optical interrogation beam to create a reflected interrogation beam and to modulate the reflected interrogation beam in response to an acoustic signal at the acoustic sensor;
an interrogation light source configured to generate the interrogation beam;
an excitation light source configured to generate an excitation beam for generating an acoustic field in a sample adjacent to the acoustic sensor;
a two-axis mirror configured to scan the interrogation beam between different locations of the acoustic sensor;
a light detector configured to detect the reflected interrogation beam from the acoustic sensor;
a controller configured to:
   use the excitation light source to generate an acoustic field in the sample, and
   detect the acoustic field at a plurality of locations of the acoustic sensor by scanning the mirror and using the light detector.

The controller may be configured to:
provide a first control signal for actuating the mirror about a first axis, and second control signal for actuating the mirror about a second axis;
scan the mirror in a fast raster scanning mode in which the first control signal is provided with a first frequency for driving the mirror in resonance about the first axis and the second control signal is provided for stepping the mirror through a plurality of angles about the second axis without resonance. The controller may be configured to operate in a Lissajous scanning mode, in which the mirror is driven to resonate about both the first and second axes.

The apparatus may further comprise a phase controller for controlling a phase difference of the interrogation beam in an optical cavity of the acoustic sensor. The controller may be configured to:
operate the phase controller to tune the phase difference of the interrogation beam in the optical cavity to a plurality of phase values;
at each phase value, use the fast raster scanning mode to direct the interrogation beam to a plurality of calibration locations of the acoustic sensor;
use the light sensor to detect the reflected interrogation beam for each phase value at each of the calibration locations;
determine, for each calibration location, a bias phase value that results in modulation of the reflected interrogation beam in response to an acoustic signal at the acoustic sensor.

The controller may be further configured to:
scan the mirror in a stepped scanning mode, in which the first control signal and the second control signal are adjusted with a frequency low enough (e.g. stepped between values at a frequency lower than the resonant frequency, for example at a rate less than half the resonant frequency) to cause the mirror to step through angles about both the first axis and second axis without resonance.

The controller may be configured to detect the acoustic field by operating the mirror in the stepped scanning mode.

In any of the aspects described herein, the interrogation beam may be a plurality of interrogation beams. The acoustic sensor may be operable to reflect each interrogation beam to create a reflected interrogation beam and to modulate each reflected interrogation beam in response to an acoustic signal at the acoustic sensor. The interrogation light source may be configured to generate each interrogation beam. The two-axis mirror may be configured to direct the interrogation beams to the acoustic sensor with the interrogation beams spaced apart from each other (e.g. with beam centres at different locations) to define a beam pattern at the acoustic sensor, and to scan the beam pattern between different locations of the acoustic sensor. The light detector may be configured to detect each reflected interrogation beam from the acoustic sensor.

The beam pattern may be widely spaced, with the distance between the two most widely separated interrogation beams at the acoustic sensor being at least 50% (or 25%) of the maximum distance between the locations at which the acoustic field at the acoustic sensor is detected during scanning of the mirror.

The photoacoustic imaging apparatus or probe head may comprise a plurality of optical fibres. The light detector may comprise a plurality of light sensors, and each of the optical fibres may be configured to form at least part of a detection optical path between the mirror and a respective light sensor.

The plurality of optical fibres may be further configured to form at least part of an interrogation optical path between the interrogation light source and the mirror.

The apparatus may comprise an optical circulator for each optical fibre, each optical circulator configured to separate the detection optical path for a respective reflected interrogation beam from an interrogation optical path for a respective interrogation beam.

The apparatus may further comprise an excitation optical fibre configured to form at least part of an optical path between the excitation light source and the acoustic sensor.

The probe head may further comprise a dichroic mirror configured to combine the interrogation beam with the excitation beam before incidence of the interrogation beam at the acoustic sensor.

The probe head may further comprise an optical relay between the mirror and the acoustic sensor.

The probe head may further comprise a tubular housing, wherein the acoustic sensor is housed within a tip of the tubular housing and the optical relay is disposed in the tubular housing.

The tubular housing may be configured for endoscopic or intra-oral use.

According to the fourth aspect, there is provided an imaging apparatus comprising:
a Fabry Perot acoustic sensor, operable to reflect an optical interrogation beam to create a reflected interrogation beam and to modulate the reflected interrogation beam in response to an acoustic signal at the acoustic sensor;
an interrogation light source configured to generate the interrogation beam;
an excitation light source configured to generate an excitation beam for generating an acoustic field in a sample adjacent to the acoustic sensor;
a visible light source configured to generate visible light and to illuminate a field of view through the acoustic sensor,
a light detector configured to detect the reflected interrogation beam from the acoustic sensor;
an image sensor configured to obtain visible light images of the field of view through the acoustic sensor, and
a controller configured to:
   use the excitation light source to generate an acoustic field in the sample, and
   detect the acoustic field using the light detector.

The imaging apparatus may further comprise any of the features defined with reference to the preceding first to third aspects, including optional features thereof.

As an alternative to visible light, other light sources may be used, for example to enable infra-red imaging (NIR, SWIR etc), photoacoustic imaging with Raman spectroscopy, and/or fluorescence imaging (e.g. using a UV source) and/or optical coherence tomography.

The visible light source may be configured to illuminate the acoustic sensor with a wide field (e.g. illuminating substantially all, or over 90% of the area of the acoustic sensor)

The imaging apparatus may further comprise an optical relay between the acoustic sensor and the image sensor.

The imaging apparatus may further comprise a tubular housing, wherein the acoustic sensor is housed within a tip of the tubular housing and the optical relay is disposed in the tubular housing.

At least part of an optical path from the visible light source to the acoustic sensor may be provided by optical fibres embedded in the walls of a tubular housing for the acoustic sensor.

The imaging apparatus may further comprise a first dichroic mirror configured to combine at least part of a visible optical path from the visible light source to the acoustic sensor with an interrogation optical path from the interrogation light source to the acoustic sensor.

The imaging apparatus may further comprise a second dichroic mirror configured to combine at least part of a excitation beam optical path from the excitation light source to the acoustic sensor with an interrogation optical path from the interrogation light source to the acoustic sensor.

The imaging apparatus may further comprise a beam splitter, configured to split light directed from the visible light source toward the acoustic sensor from visible light received through the acoustic sensor for forming an image of the field of view through the acoustic sensor.

The imaging apparatus may further comprise a beam director configured to address different locations of the acoustic sensor with the interrogation beam.

The reflected interrogation beam may be directed by the second dichroic mirror to the beam director.

The beam director may comprise a 2-axis mirror (e.g. a MEMS mirror).

### DETAILED DESCRIPTION

Embodiments of the invention will be described, purely by way of example, with reference to the accompanying drawings, in which:

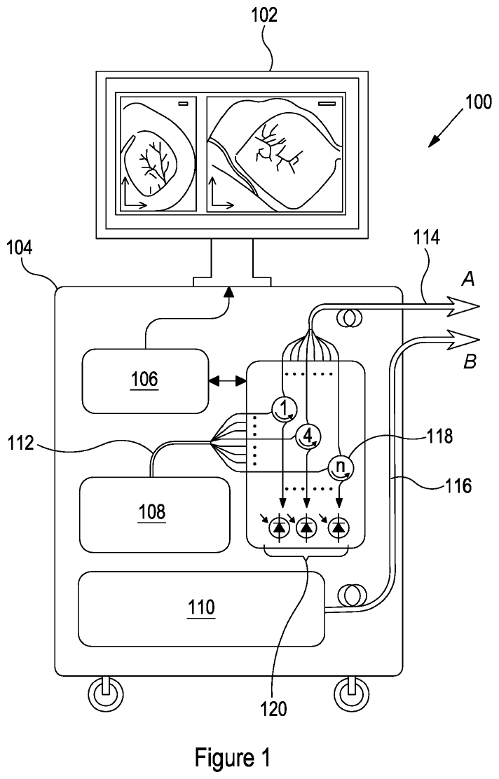

FIG. 1 is a photoacoustic imaging workstation according to an embodiment;

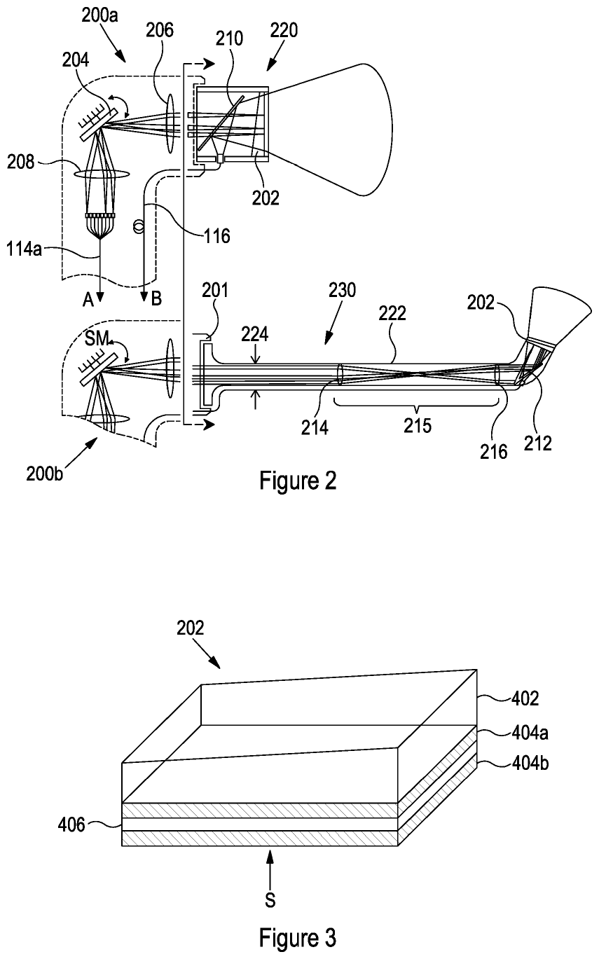

FIG. 2 is a probe head according to an embodiment, with a transcutaneous module and an intra-oral module;

FIG. 3 is a schematic of a Fabry Perot acoustic sensor;

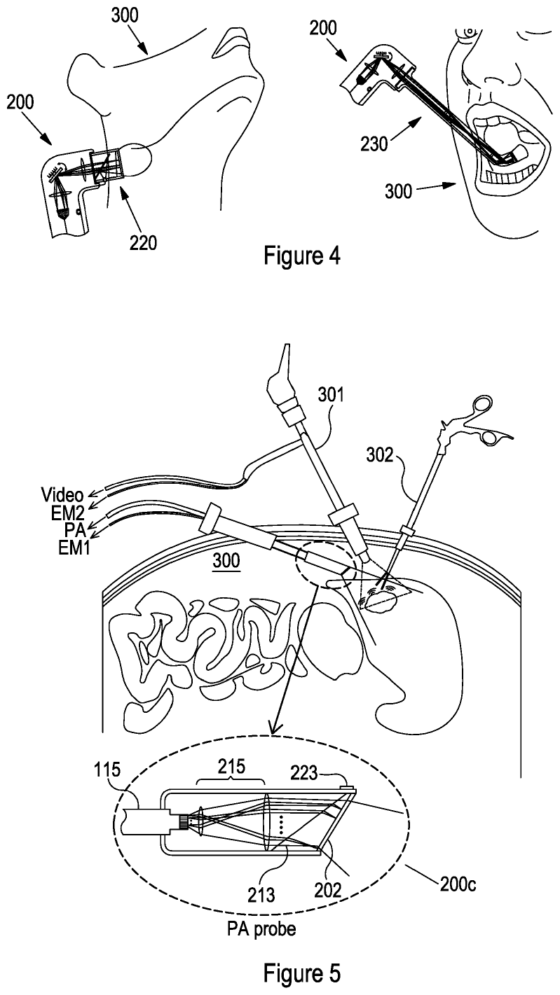

FIG. 4 is an example illustrating use of probe heads according to embodiments;

FIG. 5 illustrates a probe head according to an embodiment in the context of a laparoscopic procedure;

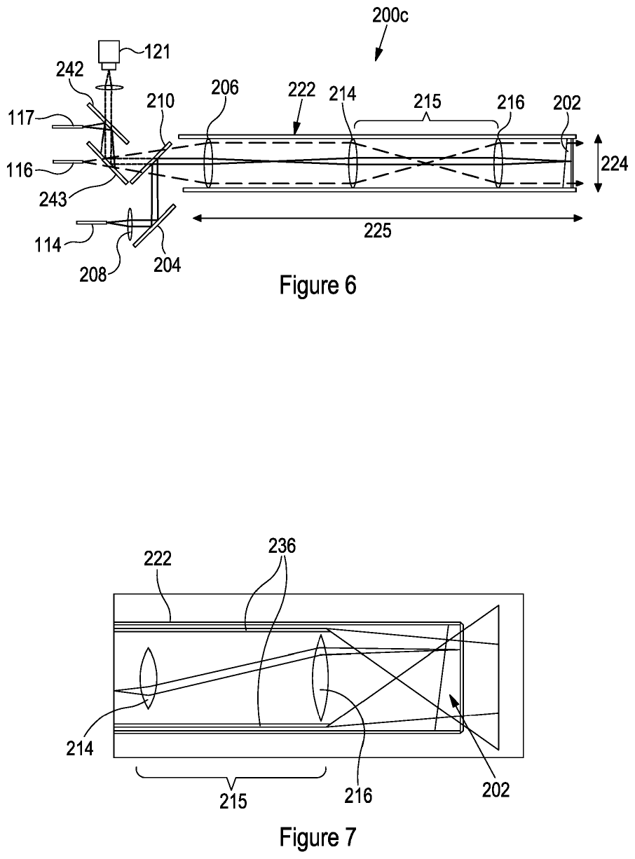

FIG. 6 is a multi-modal probe head; and

FIG. 7 shows an alternative illumination arrangement for a probe head.

FIG. 1 shows a photoacoustic imaging workstation 100, comprising a housing 104 and display screen 102. The imaging workstation 100 comprises a controller 106, interrogation light source 108, excitation light source 110, excitation optical fibre 116, interrogation optical fibre bundles 112, 114, optical circulators 118, and light detector 120. The workstation 100 may further comprise user interface devices such as a keyboard and mouse (not shown) to enable a user to control the photoacoustic imaging workstation. The controller 106 may comprise a computer/processor.

The imaging workstation 100 is configured to be connected to a photoacoustic probe head (for example, as described with reference to FIGS. 2 and 3). The imaging workstation 100 transmits excitation light and interrogation light to the probe head, and receives, from the probe head 200, reflected interrogation light. The reflected interrogation light is modulated by an acoustic field at the probe head. The reflected interrogation light is detected at the workstation by the light detector 120.

The excitation light source 110 is arranged to provide an excitation beam to be absorbed by a tissue sample adjacent to the probe head 200 (e.g. in contact therewith) and to generate a photoacoustic field in the tissue. The excitation light source 110 may provide a pulsed light output. The excitation light source 110 may be provided by an Optical Parametric Oscillator (OPO) driven by an appropriate source of coherent light, such as an Nd:YAG pulsed laser. In this way, the OPO can be used to change the excitation wavelength of the excitation light source 110 to different wavelengths that are selectively absorbed by the tissue structure, such that different structural and functional features of the tissue can be studied.

The excitation light source 110 is coupled to an excitation optical fibre 116, which is configured to transmit the excitation light beam to the probe head 200. The excitation light source 110 is coupled to controller 106 and is configured to be controlled thereby to transmit light with a selected wavelength, timing and/or power based on a control signal sent from the controller 106.

The interrogation light source 108 preferably comprises a wavelength tuneable coherent interrogation light source. In embodiments, the interrogation light source 108 may be a tuneable laser emitting coherent light in a range around 1550 nm. The interrogation light source 108 is coupled to controller 106 and is configured to be tuned thereby to transmit light at a wavelength based on a control signal sent from the controller 106.

The interrogation light source 108 in this example embodiment is coupled to a first interrogation optical fibre bundle 112, and is configured to provide an interrogation light beam to each fibre in the first interrogation optical fibre bundle 112. The use of multiple interrogation fibres enables multiple locations at the probe head 200 to be interrogated at the same time, speeding up readout times, but this is not essential. Embodiments are also envisaged in a single interrogation beam is used, and in which a single interrogation fibre couples the interrogation light source 108 to the probe head 200.

The first interrogation optical fibre bundle 112 is coupled to a second interrogation optical fibre bundle 114 by circulators 118, with each fibre in the first interrogation optical fibre 112 coupled to a corresponding fibre in the second interrogation optical fibre bundle 114 by a respective circulator 118. The circulators transmit outgoing interrogation light beams from the first interrogation optical fibre bundle 112 to the second interrogation optical fibre bundle 114.

In addition to communicating outgoing interrogation light to the probe head 200, the second interrogation optical fibre bundle 114 is configured to communicate reflected interrogation light from the probe head 200 (modulated by the acoustic field at the probe head 200) to the light detector 120. The circulators 118 are configured to direct light in the second interrogation optical fibre bundle 114 travelling from the probe head 200 to the workstation 100 to the light detector 120. The light detector 120 comprises a light sensor (e.g. a photodiode) for each optical fibre in the interrogation optical fibre bundle 112, 114. The output from the light detector 120 is provided to the controller 106 for processing, for example to enable an image of the tissue sample at the probe head 200 to be formed and displayed on the display 102. The controller 106 may be configured to process the output from the light detector 120 in any suitable way, for example to perform PA microscopy or PA tomography.

The workstation 100 may be coupled to the probe head by a flexible umbilical which includes optical fibres for communicating excitation light and interrogation light (including reflected interrogation light), and wires for communicating electrical control signals to the probe head 200.

The workstation may comprise an interrogation interface A for communicating interrogation light to the umbilical (not shown) and receiving modulated reflected interrogation light back from the umbilical. The workstation may further comprise an excitation output B for communicating excitation light to the umbilical (not shown) for transmission to the probe head 200. The umbilical may comprise a connector at the workstation and/or the probe head 200 for connecting the workstation to the probe head. In some embodiments, the umbilical may comprise a connector at both ends end, for easily connecting and disconnecting to both the workstation 100 and probe head 200. In other embodiments, the umbilical may be captive to the workstation 100 and/or the probe head 200. A connector may be provided at one end, both ends, or the umbilical may be captive to both the workstation 100 and probe head 200.

FIG. 2 shows examples of a first probe head 200a and a second probe head 200b according to example embodiments of the invention. Each probe head 200a-b comprises a MEMS mirror 204, acoustic sensor 202, interrogation optical fibre bundle 114a, excitation optical fibre 116, first lens 208, second lens 206, and dichroic mirror 210.

The probe heads 200a-b comprise an interrogation interface A configured to receive interrogation light from the workstation 100 and to transmit reflected interrogation light to the 100 workstation, and an excitation input B. The interrogation interface A and/or excitation input B may comprise fibre connectors (e.g. a multi-fibre push on connector). A single fibre connector may provide both interrogation interface A and excitation interface B.

The probe heads 200a-b in the example embodiments comprise an interrogation optical fibre bundle 114a. Each fibre of the interrogation optical fibre bundle 114a at the probe is configured to be coupled to a respective optical fibre of the second interrogation optical fibre bundle of the workstation 100. In this example, there are 16 optical fibres in the interrogation optical fibre bundle 114a, but there may be a single fibre, or a different number of fibres.

The interrogation light beams from the optical fibres of the interrogation optical fibre bundle 114a are focussed at the MEMS mirror 204 by the first lens 208. The MEMS mirror 204 reflects the interrogation light beams to be incident at the acoustic sensor 202 via the second lens 206. The first and second lenses 208, 206 may form a 4f imaging system, with divergent beams from the interrogation optical fibre bundle received at the first lens 208, the MEMS mirror 204 at a focal plane of the first and second lenses 208. 206, and focussed interrogation beams incident at the acoustic sensor 202. The pattern of interrogation fibres at their termination in the probe head 200 may thereby be reproduced at the acoustic sensor 202 at a location on the acoustic sensor that is adjustable (e.g. in x and y) by varying the tilt angles of the MEMS mirror 204 (about the first and second axes thereof).

The pattern of beams at the acoustic sensor 202 may be widely spaced, so as to reduce the amount of tilt at the MEMS mirror 204 that is required in order to interrogate an addressed region of the acoustic sensor 202. For example for interrogating a 1024×1024 array of locations at the acoustic sensor with an equal x and y pitch, 16 beams may formed into a square array, each beam spaced apart from its nearest neighbours by 256 times the pitch in both x and y. This pattern of 16 beams may be stepped from 0 to 255 times the pitch, in steps of one pitch at a time in x and y in order to sample all the 1024×1024 locations. This can be contrasted with a square block of 16 beams spaced apart by 1 pitch in x and y, which must be stepped from 0 to 1020 times the pitch in steps of four pitches at a time in x and y in order to sample all the locations. Rotating the mirror by smaller angles improves the speed of readout, makes suitable MEMS mirrors easier to source, and reduces the necessary drive voltage (tilt angle typically being approximately proportional to the square of voltage in electrostatically actuated systems). This approach also helps to linearize the control of the MEMS mirror.

The dichroic mirror 210 combines an excitation light beam, from the excitation optical fibre 116, with the interrogation light beams from the second lens 206. The acoustic sensor 202 is transparent to the excitation light beam, so that the excitation light beam can excite an acoustic field in tissue adjacent to the distal tip of the probe head 200.

The probe head 200a-b may comprise a modular arrangement, in which the MEMS mirror 204 is housed in a mirror housing, and the acoustic sensor 202 is housed in a sensor housing 220, 230 that is detachable from the mirror housing. A mechanical coupling 201 may be provided on the mirror housing and/or the sensor housing in order to allow the mirror and sensor housing to be coupled together and uncoupled (e.g. without the use of tools).

An example of this is shown in FIG. 2, with probe 200a comprising a first sensor housing 220 for transcutaneous PA imaging, and probe 200b comprising a second sensor housing 230 for intra-oral PA imaging, as shown in FIG. 4. Although FIG. 4 illustrates use of example probe heads 200a on a person, it can also be used to investigate tissue samples, ex vivo or in vitro. A similar configuration may be used for an endoscopic probe (e.g. with a flexible endoscopic tube coupled to the mirror housing).

Some embodiments may comprise more than one interchangeable sensor housing and a common mirror housing with which each sensor housing is compatible.

The second sensor housing 230 comprises a tubular section 222, with the acoustic sensor 202 placed at the distal tip thereof. The diameter 224 of the tubular section 222 is sized for convenient intra-oral use, and may be, for example 10 mm or less. The length of the tubular section 222 may be, for example, at least 10 cm. In order to extend the distance from the MEMS mirror 204 to the acoustic sensor 202, an optical relay 215 may be used, disposed within the tubular section 222. The optical relay 215 comprises a first relay lens 214 and a second relay lens 216.

The relay lenses in this embodiment are arranged to increase the working distance between the mirror 204 and the acoustic sensor 202. It is not essential that the optical relay comprise a pair of optical lenses (e.g. spherical lenses), and the optical relay may alternatively comprise rod lenses, GRIN lenses or a length of fibre bundle. In embodiments where the optical relay comprises a pair of optical lenses, the lenses do not necessarily have equal optical power. In some embodiments the optical relay may comprise a pair of lenses with different optical power. The difference in optical power may be used to control a field of view, so as to provide a different spot size at the acoustic sensor compared with the a spot size at the mirror.

In some situations, it may be helpful for the acoustic sensor 202 to point at an angle to the longitudinal axis of the tubular section 222. The example sensor housing 230 includes an angled mirror 212 to turn the optical path near the distal tip. This is not an essential feature, but may be useful. For example, the effective field of view of an angled imaging sensor can be increased by rotation of the tubular section.

FIG. 3 shows an example Fabry Perot acoustic sensor 202 comprising a wedged transparent polymer backing stub 402 on to which a multilayer sensing structure of the Fabry Perot etalon is vacuum deposited, such as by chemical vapour deposition (CVD). The structure includes a spacer 406, typically formed of Parylene polymer film 10-50 μm thick, depending upon the acoustic bandwidth required, sandwiched between two highly reflective mirrors 404a, 404b, typically provided by dichroic dielectric thin film mirrors. The spacer 406 provides the optical cavity of the Fabry Perot interferometer over which multiple reflections of light travel and from which escaping reflected light can constructively or destructively interfere depending on the phase difference between the optical fields reflected from the two mirrors of the Fabry Perot interferometer for the light in the cavity. In the embodiment shown in FIG. 3, the resonant Fabry Perot cavity is planar in geometry, but other cavity structures are suitable if they can provide a resonant effect on light in the cavity. Such alternative structures include plano-convex microresonator arrays, inverted plano-convex microresonator arrays, for example, having mirrored surfaces.

The mirrors 104a, 104b, may be designed to be highly reflective (i.e. reflects at least 95% of power) in a first wavelength range thus forming with the spacer 104 a high finesse Fabry Perot cavity in this wavelength range but highly transmissive in a second wavelength range. Preferably the first wavelength range is between 1500-1700 nm and the second wavelength range is 600 nm-1200 nm.

In use, the sensor head 100 is placed such that a sensing surface thereof S is faced against and acoustically coupled to the tissue sample to be imaged (not shown). A coupling gel may be used to keep ensure the acoustic field is conveyed from the tissue to the surface S.

The second wavelength range enables the excitation laser pulses from the excitation light source 110 to be transmitted through the acoustic sensor 202 into the tissue. The excitation light may be in the near infrared (NIR) window, where biological tissues are relatively transparent. The photoacoustic signals generated by the absorption of the light energy propagate back to the surface S where they modulate the optical thickness of the spacer 406 and thus the reflectivity of the Fabry Perot sensing structure in the 1500-1700 nm wavelength.

The MEMS mirror 204 may be electrostatically actuated, and configured to tilt about a first and second axis in response to respective first and second control signals. The MEMS mirror 204 may include a mechanical layer from which the mirror element and supporting springs are formed. The supporting springs may be configured to enable rotation about a first and second rotation axis, without contact between bearing surfaces (i.e. on compliant springs). The mechanical layer may be patterned using lithographic methods, for example by photolithographic pattern transfer and etching. The mechanical layer may comprise a semiconductor material. The resonant frequency of the MEMS mirror 204 in rotation about the first and second axis may be at least 100 Hz, or at least 250 Hz. The diameter of the MEMS mirror may be less than 1 cm, or less than 5 mm. The mechanical layer may be anchored to a semiconductor substrate such as a silicon substrate.

The MEMS mirror 204 may be operable in two different scan modes about each rotation axis: resonant, or step-scanned. In resonant scanning, the MEMS mirror 204 is driven to resonate about the respective axis or axes, by using a control signal at or near the resonant frequency of a vibration about the respective axis. This can be used to scan a Lissajous figure of the acoustic sensor 202 at high speed. In step-scanning, the MEMS mirror 204 is driven by a control signal with a frequency below the resonant frequency, so that the MEMS mirror 204 tracks the control signal, substantially without oscillation. For example, the control signal may be stepped through a sequence of values at a frequency of less than half the resonant frequency to perform a slow raster scan.

A fast raster mode of scanning can be defined, in which the MEMS mirror is driven in rotation about one axis in resonant mode, and about the other axis in step-scanned mode. Scanning in this way is slower than using both axis in resonant mode, but this approach makes calibrating the response of the scanner more straightforward, since the relationship between the driving voltage of the control signal and the deflection angle is very non-linear in many MEMS mirrors (e.g. where electrostatic actuation is used).

In order to provide sufficient sensitivity to acoustic signals, the finesse of the acoustic sensor's etalon transfer function may be relatively high. This high finesse makes it important that the wavelength of the interrogation beam is matched to the optical thickness of the cavity 406. This can be thought of in terms of an optimal phase difference between the incident and reflected wave in the etalon. In order to ensure the acoustic sensor 202 modulates the interrogation beam in response to an acoustically induced changes in thickness, the phase difference may be adjusted by varying the optical thickness of the cavity or the wavelength of the interrogation beam.

The optical thickness of the cavity 406 may be adjusted by varying a refractive index of a material in the cavity (e.g. a liquid crystal material), or by controlling a thickness of the cavity using thermal expansion and contraction (e.g. by controlling its temperature with the excitation beam). The adjustment required to achieve the required phase difference may be termed the bias phase.

The thickness of the cavity 406 (as fabricated) may vary over the area of the acoustic sensor 202, with the result that different locations of the acoustic sensor 202 have different appropriate bias phase. In order to calibrate the bias phase, the acoustic sensor 202 may be interrogated over a range of phase difference values (e.g. interrogation beam wavelength or cavity optical thickness) at each location. The reflected interrogation beam (or beams) may be used to infer an etalon transfer function (e.g. etalon reflectance vs interrogation beam wavelength) at each of a plurality of calibration locations. The appropriate bias phase for each location may be inferred from the etalon transfer function (e.g. a point of maximum slope). The calibration locations may be a subset of the locations at which measurements are acquired during imaging, since the thickness of the cavity may vary with a low spatial frequency.

The settling time for varying the phase difference may be relatively slow (e.g. the settling time for a wavelength adjustable source). The speed of obtaining the calibration bias phases may therefore be enhanced by scanning each calibration location at a specific phase difference (e.g. wavelength of the interrogation light), then adjusting the phase difference, and then re-scanning each calibration location. This can be repeated until enough values of phase difference have been obtained for each calibration location to determine the bias phase for each calibration location.

The applicant has found that a fast raster scanning mode (in which the MEMS mirror is scanned about one axis in a resonant mode, and about the other axis in step-scanned mode) is advantageous for obtaining the calibration data (comprising the bias phase at each calibration location),In other embodiments, both axes may be driven in resonance to obtain calibration data, which may be faster (but potentially more costly to implement).

The pulse repetition frequency for the excitation laser (e.g. <1 kHz) may be a limiting factor for the speed of readout, so signal acquisition (for imaging) may be carried out by scanning both axes of the MEMS mirror in a step-scan mode.

FIG. 5 illustrates a PA probe 200c in the context of a laparoscopic procedure, in which a surgical tool 302 is used in conjunction with a PA probe 200c and a video endoscope 301. In this example the PA probe 200c comprises a relay optic 215 that is configured to vary the width of the interrogation beam, from a narrow beam at the fibre bundle 115, to a wider beam at the acoustic sensor 202. A refractive prism 213 is provided for refracting the interrogation optical beams so as to provide an angled field of view of the acoustic sensor 202 (with respect to the beams as they exit from the optical fibre bundle 115). The MEMS mirror (not shown in FIG. 5) in this example may scan at least one interrogation beam to different fibres of the fibre bundle 115 in order to scan different locations of the acoustic sensor 202.

FIG. 6 schematically illustrates a probe head 200c that facilitates both PA imaging and obtaining visible light video. The probe head 200c comprises: interrogation fibre bundle 114, excitation fibre 116, visible light source 117, visible light camera 121, tubular housing 222, optical relay 215, focussing lenses 208, 206, acoustic sensor 202, dichroic mirrors 210, 243, beam splitter 242, and MEMS mirror 204.

The photoacoustic imaging for this probe head 200c works in a similar way as described with reference to the probe head 200b of FIG. 2. Parts with like reference numerals perform essentially the same functions in FIG. 7. The dichroic mirror 210 in this embodiment (for combining the optical paths of the interrogation and illumination light sources) is nearer to the proximal tip of the tubular housing 222 than the distal tip. The MEMS mirror 204 again steers the interrogation beams from the interrogation fibre bundle 114 to different locations of the acoustic sensor 202, with focussing lenses 208, 206 respectively disposed between the MEMS mirror 204 and the interrogation fibre bundle and the MEMS mirror 204 and the acoustic sensor 202.

The dichroic mirror 243 combines the visible illumination light beam from the visible light source 117 with the excitation light beam from the excitation fibre 116. The visible light thereby at shares a common illumination optical path with the excitation beam to the acoustic sensor 202. The acoustic sensor 202 is transparent to the visible light, so a field of view in front of the acoustic sensor is thereby illuminated, and light reflected from this field of view is received along the same optical path back to the beam splitter 242, which provides a portion of the reflected light to the camera 121, thereby allowing both visible light video and PA imaging to be performed with a single probe head. The camera 121, visible light source 117 and beam splitter 242 may alternatively be located remote from the probe head 200c (for example in the workstation 100), and a fibre bundle used to communicate the visible light to the probe head and the reflected visible light back to the camera 121. The same fibre bundle could be used to deliver visible light, interrogation light and excitation light.

Other illumination arrangements are also possible. For example, it is not necessary for the visible illumination to share an optical path with the excitation beam, although this is convenient, and may result in a more compact device.

FIG. 7 illustrates an alternative illumination optical arrangement for a probe head, in which illumination optical fibres 236 are disposed at the periphery of the tubular housing 222 (e.g. at least partially embedded therein). The end face of these illumination optical fibres 236 may be polished at an angle, to facilitate more uniform illumination of the acoustic sensor 202. The illumination optical fibres 236 may be used as an optical path for the visible light and/or the excitation light beam.

The use of a 2 axis mirror 204 (e.g. a MEMS mirror) to address different locations of the acoustic sensor 202 provides significant advantages over prior art arrangements which employ conventional galvanometer based scanning mirrors. Such galvo-mirrors typically have only one axis of tilt, meaning that two galvo mirrors are required to scan in x and y at the acoustic sensor. Compared with a conjugate arrangement of two single-axis (non-MEMS) galvo-mirrors, a 2-axis mirror (such as a 2-axis MEMS mirror) is significantly more compact, lightweight and utilises a simpler optical arrangement (with no need for the concave mirror pair that are typically used for a conjugate pair of 1D galvo scanners). It also offers advantages in terms of accuracy, with the scan region (spot size and field of view) being less distorted (particularly for multi-beam arrangements), since the beam is generally pivoted about a single point with a MEMS mirror.

Although specific examples have been shown in which fibre coupling is used to route optical signals, it will be understood that this is not essential, and any of the fibre coupled optical arrangements may be replaced with free-space equivalents. Although embodiments have been described with more than one interrogation beam/optical fibre, it will be readily understood that a single interrogation beam/optical can be used in alternative embodiments. Other such variations are also within the scope of the invention, which should be determined with respect to the appended claims.

### REFERENCES

Zhang E, Laufer J, Beard P. Backward-mode multiwavelength photoacoustic scanner using a planar Fabry-Perot polymer film ultrasound sensor for high-resolution three-dimensional imaging of biological tissues. Applied Optics. 2008; 47(4):561-577

Huynh N, Ogunlade O, Zhang E, Cox B, Beard P. Photoacoustic imaging using an 8-beam Fabry-Perot scanner. Photons Plus Ultrasound: Imaging and Sensing 2016: SPIE

Laufer J, Johnson P, Zhang E, Treeby B, Cox B, Pedley B, Beard P. In vivo preclinical photoacoustic imaging of tumor vasculature development and therapy. Journal of Biomedical Optics. 2012; 17(5):56016-1-56016-8.

Beard, P. (2011). Biomedical photoacoustic imaging. Interface Focus, 1(4), 602-631

Klann, M., & Koch, C. (2005). Measurement of spatial cross sections of ultrasound pressure fields by optical scanning means. IEEE Transactions on Ultrasonics, Ferroelectrics, and Frequency Control, 52(9), 1546-54. Retrieved from http://www.ncbi.nlm.nih.gov/pubmed/16285453

Ansari, R., Zhang, E. Z., Desjardins, A. E., & Beard, P. C. (2017). All-optical forward-viewing endoscopic probe for high resolution 3D photoacoustic tomography. In Proc SPIE (Vol. 10064, pp. 1-6). https://doi.org/10.1117/12.2250617

Ansari, R., Zhang, E. Z., Desjardins, A. E., & Beard, P. C. (2018). All-optical forward-viewing photoacoustic probe for high-resolution 3D endoscopy. Light: Science & Applications, 7(1), 75. https://doi.org/10.1038/s41377-018-0070-5

Zhang, E. Z., Povazay, B., Laufer, J., Alex, A., Hofer, B., Pedley, B., . . . Drexler, W. (2011). Multimodal photoacoustic and optical coherence tomography scanner using an all optical detection scheme for 3D morphological skin imaging. Biomedical Optics Express, 2(8), 2202-2215.

What is claimed is:

1. A photoacoustic probe head, comprising:
a Fabry Perot acoustic sensor, operable to reflect an optical interrogation beam to create a reflected interrogation beam and to modulate the reflected interrogation beam in response to an acoustic signal at the acoustic sensor;
an interrogation interface configured to receive an interrogation beam, and to receive the reflected interrogation beam from the acoustic sensor;
an excitation input configured to receive an excitation beam for generating an acoustic field in a sample adjacent to the acoustic sensor;
a two-axis mirror configured to scan the interrogation beam between different locations of the acoustic sensor.

2. The photoacoustic probe head of claim 1, wherein the two-axis mirror is further configured to direct the reflected interrogation beam to the interrogation interface.

3. The photoacoustic probe head of claim 1, further comprising:
a mirror housing within which the mirror is disposed, and
a sensor housing with the acoustic sensor at a distal tip thereof,
wherein the mirror housing and sensor housing together comprise a mechanical coupling for removably attaching the sensor housing to the mirror housing.

4. (canceled)

5. A photoacoustic imaging apparatus, comprising:
the probe head of claim 1,
an interrogation light source configured to generate the interrogation beam and coupled to the interrogation input;
an excitation light source configured to generate an excitation beam for generating an acoustic field in a sample adjacent to the acoustic sensor, the excitation light source coupled to the excitation input;
a light detector coupled to the interrogation port and configured to detect the reflected interrogation beam from the acoustic sensor;
a controller configured to:
use the excitation light source to generate an acoustic field in the sample, and
detect the acoustic field at a plurality of locations of the acoustic sensor by scanning the mirror and using the light detector.

6. The photoacoustic imaging apparatus of claim 5, wherein the probe head is connected to the interrogation light source and the excitation light source by a flexible umbilical.

7. (canceled)

8. The photoacoustic imaging apparatus of claim 5, wherein the controller is configured to:
provide a first control signal for actuating the mirror about a first axis, and second control signal for actuating the mirror about a second axis;
scan the mirror in a fast raster scanning mode in which the first control signal is provided with a first frequency for driving the mirror in resonance about the first axis and the second control signal is provided for stepping the mirror through a plurality of angles about the second axis without resonance.

9. The photoacoustic imaging apparatus of claim 8, further comprising:
a phase controller for controlling a phase difference of the interrogation beam in an optical cavity of the acoustic sensor;
wherein the controller is configured to:
operate the phase controller to tune the phase difference of the interrogation beam in the optical cavity to a plurality of phase values;
at each phase value, use the fast raster scanning mode to direct the interrogation beam to a plurality of calibration locations of the acoustic sensor;
use the light sensor to detect the reflected interrogation beam for each phase value at each of the calibration locations;
determine, for each calibration location, a bias phase value that results in modulation of the reflected interrogation beam in response to an acoustic signal at the acoustic sensor.

10. The photoacoustic imaging apparatus of claim 8, wherein the controller is further configured to:
scan the mirror in a stepped scanning mode, in which the first control signal and the second control signal are adjusted with a frequency low enough to cause the mirror to step through angles about both the first axis and second axis without resonance, and the controller is further configured to detect the acoustic field by operating the mirror in the stepped scanning mode.

11. (canceled)

12. The photoacoustic imaging apparatus of claim 5, wherein the interrogation beam is a plurality of interrogation beams, wherein:
the acoustic sensor is operable to reflect each interrogation beam to create a reflected interrogation beam and to modulate each reflected interrogation beam in response to an acoustic signal at the acoustic sensor;
the interrogation light source is configured to generate each interrogation beam;
the two-axis mirror is configured to;
direct the interrogation beams to the acoustic sensor with the interrogation beams spaced apart from each other to define a beam pattern at the acoustic sensor, and
scan the beam pattern between different locations of the acoustic sensor;
the light detector is configured to detect each reflected interrogation beam from the acoustic sensor; and the beam pattern is widely spaced, with the distance between the two most widely separated interrogation beams at the acoustic sensor being at least 50% of the maximum distance between the locations at which the acoustic field at the acoustic sensor is detected during scanning of the mirror.

13. (canceled)

14. The photoacoustic imaging apparatus of claim 12, further comprising a plurality of optical fibres, wherein the light detector comprises a plurality of light sensors, and each of the optical fibres is configured to form at least part of a detection optical path between the mirror and a respective light sensor; and the plurality of optical fibres is further configured to form at least part of an interrogation optical path between the interrogation light source and the mirror.

15-18. (canceled)

19. The photoacoustic probe head of claim 1, further comprising an optical relay between the mirror and the acoustic sensor; and further comprising a tubular housing, wherein the acoustic sensor is housed within a tip of the tubular housing and the optical relay is disposed in the tubular housing

20. (canceled)

21. The photoacoustic imaging apparatus or probe head of claim 19, wherein the tubular housing is configured for endoscopic or intra-oral use.

22. An imaging apparatus comprising:
a Fabry Perot acoustic sensor, operable to reflect an optical interrogation beam to create a reflected interrogation beam and to modulate the reflected interrogation beam in response to an acoustic signal at the acoustic sensor;
an interrogation light source configured to generate the interrogation beam;
an excitation light source configured to generate an excitation beam for generating an acoustic field in a sample adjacent to the acoustic sensor;
a visible light source configured to generate visible light and to illuminate a field of view through the acoustic sensor,
a light detector configured to detect the reflected interrogation beam from the acoustic sensor;
an image sensor configured to obtain visible light images of the field of view through the acoustic sensor, and
a controller configured to:
use the excitation light source to generate an acoustic field in the sample, and
detect the acoustic field using the light detector.

23. The imaging apparatus of claim 22, further comprising an optical relay between the acoustic sensor and the image sensor and further comprising a tubular housing, wherein the acoustic sensor is housed within a tip of the tubular housing and the optical relay is disposed in the tubular housing

24. (canceled)

25. The imaging apparatus of claim 23, wherein at least part of an optical path from the visible light source to the acoustic sensor and/or from the excitation light source to the acoustic sensor is provided by optical fibres embedded in the walls of the tubular housing for the acoustic sensor.

26. The imaging apparatus of claim 22, further comprising a first dichroic mirror configured to combine at least part of a visible optical path from the visible light source to the acoustic sensor with an interrogation optical path from the interrogation light source to the acoustic sensor.

27. The imaging apparatus of claim 22, further comprising a second dichroic mirror configured to combine at least part of a excitation beam optical path from the excitation light source to the acoustic sensor with an interrogation optical path from the interrogation light source to the acoustic sensor.

28. The imaging apparatus of claim 22, further comprising a beam splitter, configured to split light directed from the visible light source toward the acoustic sensor from visible light received through the acoustic sensor for forming an image of the field of view through the acoustic sensor.

29. The imaging apparatus of claim 22, further comprising a beam director, comprising a 2-axis mirror, configured to address different locations of the acoustic sensor with the interrogation beam.

30. The imaging apparatus of claim 29, further comprising a second dichroic mirror configured to combine at least part of a excitation beam optical path from the excitation light source to the acoustic sensor with an interrogation optical path from the interrogation light source to the acoustic sensor, wherein the reflected interrogation beam is directed by the second dichroic mirror to the beam director.

31. (canceled)
